(19) United States
(12) Patent Application Publication
(10) Pub. No.: US 2024/0124379 A1
(43) Pub. Date: Apr. 18, 2024

(54) HYDROXYLATION OF ALKANES USING OZONE

(71) Applicant: University of Kansas, Lawrence, KS (US)

(72) Inventors: Bala Subramaniam, Lawrence, KS (US); Hongda Zhu, Lawrence, KS (US); Timothy A. Jackson, Lawrence, KS (US)

(21) Appl. No.: 18/278,329

(22) PCT Filed: Mar. 8, 2022
(86) PCT No.: PCT/US22/71022

§ 371 Date: Aug. 22, 2023

Related U.S. Application Data
(60) Provisional application No. 63/158,522, filed on Mar. 9, 2021.

(51) Int. Cl. C07C 29/48

(52) U.S. Cl. CPC C07C 29/48

(57) ABSTRACT

Processes for oxidizing alkanes are provided. In embodiments, such a process comprises combining an alkane, e.g., isobutane, and ozone in a liquid phase medium comprising a protic additive, e.g., water, under conditions sufficient to oxidize the alkane to products comprising a hydroxylate.

### CROSS-REFERENCE TO RELATED APPLICATIONS

The present application claims priority to U.S. provisional patent application number 63/158,522 that was filed Mar. 9, 2021, the entire contents of which are incorporated herein by reference.

### REFERENCE TO GOVERNMENT RIGHTS

This invention was made with government support under 1922649 awarded by the National Science Foundation. The government has certain rights in the invention.

### BACKGROUND

Isobutane is an important commodity chemical precursor, conventionally produced in refineries via cracking and isomerization. Natural gas liquids, including those extracted along with shale gas, provide a major alternate source for isobutane. Isobutane is primarily used as a feedstock to produce tent-butyl alcohol (TBA) and tent-butyl hydroperoxide (TBHP) by oxidation with O₂. Isobutylene, produced via either isobutane dehydrogenation or TBA dehydration, is an important alkylating reagent for production of chemical and pharmaceutical precursors. Isobutylene is also a monomer for the production of synthetic resins and butyl rubber.

Industrial isobutane oxidation uses O₂ as oxidant and is performed at 120-140° C. and 2.5-3.5 MPa, with ˜3-10% O₂ in the gas phase. At these conditions, isobutane exists as a liquid and O₂ is bubbled through the liquid phase. The vapor phase is typically maintained above the upper flammability limit for isobutane. Free radical initiators such as TBHP and di-tent-butyl peroxide (DTBP) are usually co-fed to reduce the induction period. At these conditions, the isobutane conversion is ˜4-8% per hour, producing nearly equimolar amounts of TBA and TBHP at approximately 96% selectivity. The TBHP is used as an oxidant for propylene oxidation to propylene oxide, producing TBA as coproduct.

For isobutane oxidation with O₂, the initiation mechanisms in the liquid phase and gas phase are similar. However, the product selectivities strongly depend on the relative concentrations of the substrate and oxidant. Compared to liquid phase oxidation, isobutane oxidation in a gas phase requires longer induction periods even at 260-360° C. and produces a wider spectrum of products. This is caused by side reactions, including over-oxidation of terminal carbons and C—C bond cleavage.

### SUMMARY

Provided herein are processes for oxidizing alkanes in the liquid phase using ozone. To illustrate the present processes, the Examples, below, demonstrate isobutane ozonation to tent-butyl alcohol (TBA) with 85%-90% selectivity in a biphasic gas-liquid process at ambient temperatures (16-18.5° C.) and mild pressures (˜0.6 MPa). The observed TBA selectivity is much higher than the value reported for either gas-phase ozonation or air oxidation in liquid isobutane. In addition, it is found that O₃ utilization (defined as moles of TBA produced per mole of ozone consumed) to form TBA is maximized (˜1.5 moles TBA/mole O₃ consumed) by adding certain amounts of protic compounds (e.g., water or alcohols) in the reaction mixture, and by maintaining a certain O₃ partial pressure in the gas phase that limits the liquid phase O₃ concentration to avoid O₃ decomposition. The relatively low CO₂ formation (<6% selectivity at optimal conditions) suggests that TBA formation occurs predominantly via hydrotrioxide formation with high atom economy.

In embodiments, a process for oxidizing an alkane comprises combining an alkane and ozone in a liquid phase medium comprising a protic additive under conditions sufficient to oxidize the alkane to products comprising a hydroxylate.

In embodiments, a process for oxidizing an alkane comprises combining an alkane and ozone in a liquid phase medium comprising a protic additive under conditions sufficient to oxidize the alkane to products comprising a hydroxylate, wherein the liquid phase medium is free of added CO₂, and adding the protic additive to the liquid phase medium prior to oxidizing the alkane.

Other principal features and advantages of the disclosure will become apparent to those skilled in the art upon review of the following drawings, the detailed description, and the appended claims.

### BRIEF DESCRIPTION OF THE DRAWINGS

Illustrative embodiments of the disclosure will hereafter be described with reference to the accompanying drawings.

FIG. 1 shows temporal product profiles from batch reactor experiments of isobutane ozonation (reaction conditions other than batch time are identical to those provided in Table 2).

FIG. 2 shows the effect of initial water quantity on product distribution and O₃ utilization. Conditions: T=18.5° C., 5% O₃/(O₃+O₂+N₂), P_{iC4}=0.30 MPa, P_{O3+O2+N2}=0.34 MPa.

FIG. 3 shows the effect of non-condensable gas partial pressure on product distribution and ozone utilization. Conditions: T=18.5° C., P_{iC4}=0.30 MPa, 5% O₃/(O₃+O₂+N₂), initial H₂O quantity=5.53 mmol. Other byproducts in small quantities: TBHP selectivity ˜0.01%, hydroxyacetone, and one unidentified.

FIG. 4 shows the effect of O₃/(O₃+O₂+N₂) mole fraction on product distribution and O₃ utilization. Conditions: T=18.5° C., P_{iC4}=0.30 MPa, P_{O3+O2+N2}=0.34 MPa, initial H₂O quantity=5.53 mmol.

FIG. 5 shows the effect of initial TBA addition on product distribution and O₃ utilization. Conditions: T=18.0° C., P_{iC4}=0.29 MPa, 5% O₃/(O₃+O₂+N₂), P_{O3+O2+N2}=0.34 MPa, no initial H₂O addition.

FIG. 6, pathways a, b₁, b₂, c, d₁, d₂, b_{OH}, d_{OH}, shows reaction pathways of isobutane ozonation (the dominant pathway is marked).

FIG. 7 shows a schematic of the reactor setup for carrying out the present methods.

FIG. 8 shows plausible over-oxidation pathways of the oxygenate products.

FIG. 9 shows decomposition of oxygen-rich intermediate radicals.

### DETAILED DESCRIPTION

The present disclosure provides processes for oxidizing alkanes in the liquid phase using ozone. By “liquid phase” it is meant that the relevant reactions take place in the liquid phase, as opposed to the gas phase. Ozone is a useful oxidant for hydrocarbons, but significant challenges exist related to its effective utilization. For example, ozone decomposes quickly in the liquid phase. Moreover, intermediate compounds are often much more reactive towards ozone than the starting alkane. Thus, a key challenge in liquid phase ozonation of alkanes is the optimization of ozone utilization towards the desired reaction rather than substrate over-oxidation and ozone decomposition.

In embodiments, a process for oxidizing an alkane includes combining an alkane with ozone in a liquid phase medium. The ozone may be provided as a feed gas mixture, e.g., O₃ in O₂ or O₃ in air. An inert gas, e.g., N₂, Ar, etc., may be included in the feed gas mixture. This is useful to keep a vapor phase present above the liquid phase medium outside of its flammability envelope at the temperature and pressure being used in the process. The liquid phase medium generally also comprises a protic additive. This feature is derived from the serendipitous finding that the use of the protic additive can lead to unexpectedly high selectivity for a hydroxylate product as well as unexpectedly high ozone utilization. By “hydroxylate product,” it is meant the product corresponding to the alkane undergoing the oxidative conversion with a single C—H bond of the alkane replaced with a single C—OH bond. These findings are illustrated in the Examples, below, for the oxidation of isobutane to the hydroxylate product, tent-butyl alcohol, with 85% to 90% selectivity at mild temperatures and pressures.

The present processes may be used with a variety of alkanes. The alkane may be linear, branched, or cyclic. In embodiments, the alkane is linear or branched, and not cyclic. The alkane may include various numbers of carbons, e.g., one, two, three, four, five, six, seven, eight, etc. In embodiments, the alkane is a C1-C20 alkane, a C1-C15 alkane, a C4-C20 alkane, a C4-C18 alkane, a C4-C16 alkane, a C4-C15 alkane, or a C4-C12 alkane. Specific illustrative alkanes include methane, propane, n-butane, and isobutane. However, in embodiments, the alkane is not methane. In embodiments, the liquid phase medium does not comprise methane. Combinations of different types of alkanes may be used. In the present processes, the alkane is generally in its liquid form (i.e., a liquid at the temperature and pressure used during the oxidative conversion).

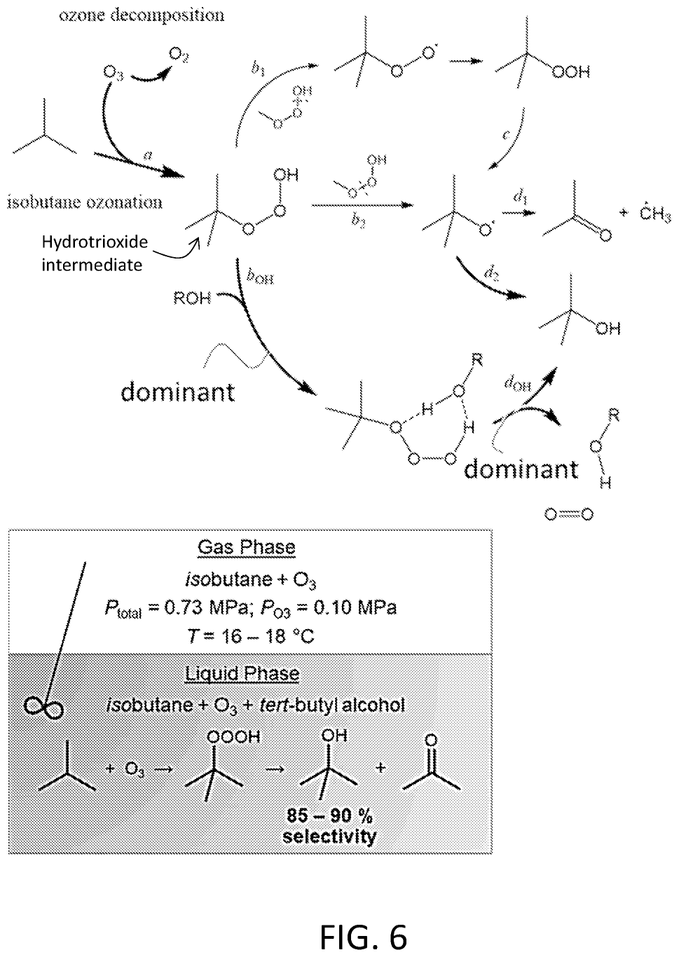

Similarly, the present processes may be used with a variety of protic additives. The protic additive is a compound that is capable of hydrogen bonding by comprising at least one hydrogen atom bound to an electronegative atom. Without wishing to be bound to any particular theory, it is believed that the protic additive stabilizes a hydrotrioxide intermediate generated during the oxidative conversion reaction. (See FIG. 6.) Thus, desirably, the protic additive is capable of stabilizing such a hydrotrioxide intermediate via hydrogen bonding. The protic additive is generally one that is a liquid at the temperature and pressure used during the oxidative conversion. In embodiments, the protic additive has a pKₐ in water at room temperature (20° C. to 25° C.) of greater than 3, 4, 5, 6, 7, 8, 9, 10, etc. Illustrative protic additives include water and alcohols. The alcohol may be a short chain alcohol having from 1 to 6 carbons, from 1 to 5 carbons, or from 1 to 4 carbons, e.g., methanol, ethanol, isopropanol, tent-butyl alcohol. In embodiments, the protic additive is not a carboxylic acid such as formic acid, acetic acid, propionic acid, or butyric acid; in such embodiments, the liquid phase medium is free of these components. In embodiments, the protic additive is not a strong acid such as sulfuric acid or a super acid having an acidity stronger than sulfuric acid; in such embodiments, the liquid phase medium is free of these components. The protic additives are desirably distilled to eliminate the presence of certain acidic impurities; in such cases, the protic additive may be referred to as a “distilled protic additive.” Combinations of different types of protic additives may be used.

The protic additive generally refers to a compound which is added to the liquid phase medium prior to carrying out the oxidative conversion. This is by contrast to the protic additive being a product of the oxidative conversion reaction (e.g., H₂O or a hydroxylate product). In embodiments, the protic additive is not a product of the oxidative conversion reaction. In embodiments, the protic additive is not a hydroxylate product, e.g., tent-butyl alcohol, of the oxidative conversion reaction. In embodiments, the present process comprises adding any of the disclosed protic additives to the liquid phase medium prior to the oxidative conversion to produce a product from the alkane.

Although no catalyst is required for the present processes, in other embodiments, a catalyst may be used. If a catalyst is used, the catalyst may contain transition metals or combinations, such as a first-row transition metal (e.g., Fe, Co, Ni) or a platinum group metal, e.g., Pd, Pt. The catalyst may be supported transition metal catalyst in which the selected transition metal is incorporated into or deposited on a surface of a metal oxide substrate, e.g., an oxide of aluminum, silicon, titanium, magnesium, cerium, zirconium, etc. Illustrative metal oxide substrates include silicate and zeolite. Mesoporous silicates such as KIT-5, KIT-6, SBA-16, TUD-1 may be used.

As described above, the oxidative conversion reaction is carried out in the liquid phase medium. The liquid phase medium comprises the selected alkane and the ozone. As noted above, the alkane itself is generally a liquid at the temperature and pressure used during the present processes. Generally, an excess amount of the alkane is used. The liquid alkane solubilizes at least some of the ozone therein. The liquid phase medium generally further comprises any of the protic additives as described above. The catalyst, if present, may either be dissolved or suspended in the liquid phase medium. In embodiments, the liquid phase medium comprises one or more alkanes, ozone, one or more protic additives, and optionally, a catalyst. However, such embodiments also encompass the presence of the resulting products of the oxidative conversion in the liquid phase medium.

By contrast to some existing oxidative conversion processes involving hydrocarbons and oxidants such as ozone, the present processes do not require the use of carbon dioxide (CO₂). Thus, at least in embodiments, the liquid phase medium is free of, i.e., does not comprise, CO₂ (whether the CO₂ is in its gaseous or liquid state). These embodiments refer to the absence of any CO₂ (whether the CO₂ is in its gaseous or liquid state) separately added to the liquid phase medium. These embodiments, however, do not preclude the formation of an amount of CO₂ as a product of the oxidative conversion. However, if any CO₂ is present in the liquid phase medium as a product of the oxidative conversion, it is generally present at an amount of less than 1 mmol. This includes less than 0.8 mmol, less than 0.6 mmol, less than 0.4 mmol, less than 0.2 mmol, and less than 0.1 mmol. This includes from 0.001 mmol to 0.8 mmol.

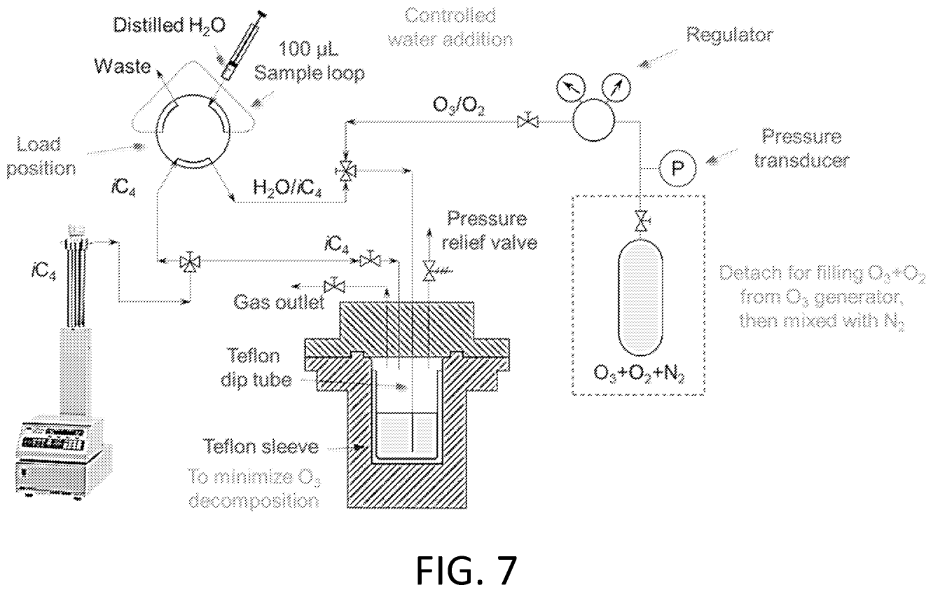

The present processes may be carried out in a variety of reactor systems, including batch reactor systems, semi-continuous flow reactor systems, and continuous flow reactor systems. FIG. 7 shows a schematic of an illustrative batch reactor system which may be used. However, as described further in the Examples, below, reactor systems in which gas (e.g., feed gas mixture) and liquid (e.g., alkane) phases may be continuously admitted and withdrawn are useful to maximize O₃ utilization and yield of hydroxylate products. It is noted that for semi-continuous and continuous reactor systems, the addition of the protic additive prior to the oxidative conversion may refer to an initial addition of the protic additive. In such embodiments, subsequent to the initial addition, the protic additive may be continuously or intermittently added to the liquid phase medium. In other such embodiments, subsequent to the initial addition, no further protic additive is added to the liquid phase medium.

The conditions used in the present processes to induce oxidative conversion refer to parameters such as the temperature, the total pressure, as well as others further described below. The temperatures and total pressures used are generally mild. In embodiments, the temperature is at least 15° C. Illustrative temperatures include those in a range of from 15° C. to 60° C., 15° C. to 50° C., 15° C. to 25° C., 15° C. to 20° C., and 16° C. to 19° C. The total pressure refers to the total pressure of the vapor phase present above, and in contact with, the liquid phase medium at the selected temperature. This vapor phase comprises ozone, as well as any other gases present in the gas mixture used to deliver the ozone, e.g., O₂, air, an inert gas such as N₂, or combinations thereof. The vapor phase also generally comprises some amount of the vapor form of the selected alkane. In embodiments, the total pressure is less than 5 MPa, less than 3 MPa, or less than 1 MPa. Illustrative total pressures include those in a range of from 0.45 MPa to 5 MPa, from 0.45 MPa to 3 MPa, and from 0.45 MPa to 1 MPa. These pressures are significantly lower than those used in ozonolysis reactions using liquid CO₂ or CO₂ expanded liquids.

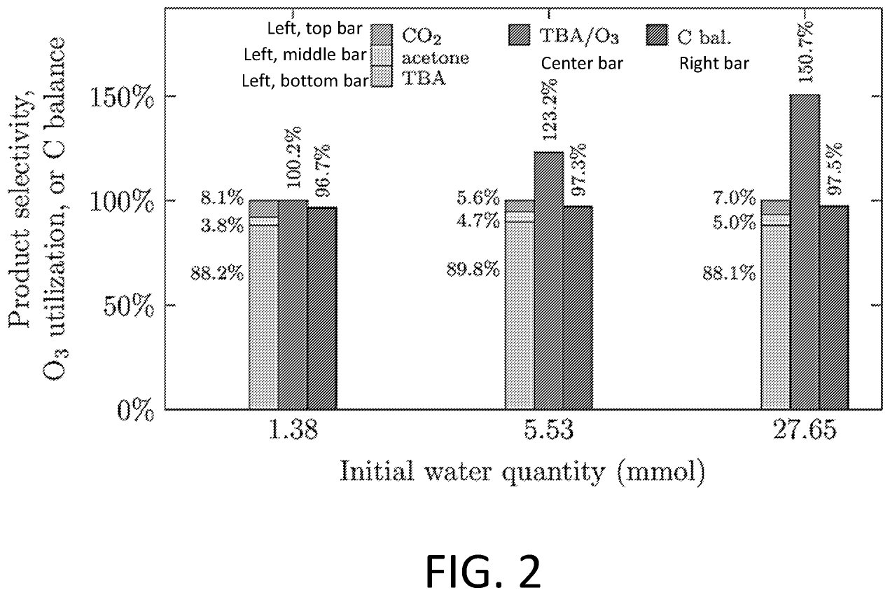

The conditions also refer to the amount of a protic additive in the liquid phase medium. The amount is generally selected to maximize O₃ utilization, which may be quantified as the number of moles of a desired product (e.g., hydroxylate product)/moles of ozone fed. Illustrative amounts of protic additive include at least 0.5 mmol, at least 1 mmol, at least 10 mmol, or at least 20 mmol. In embodiments, the amount is in a range of from 0.5 mmol to 100 mmol or 1 mmol to 100 mmol. The amount of the protic additive in the liquid phase medium may also be referred to as a volume % of the total volume of the liquid phase medium. In embodiments, the amount is in a range of from 0.2 volume % to 5 volume %, from 0.2 volume % to 2 volume %, from 0.2 volume % to 1.5 volume %, or from 0.2 volume % to 1 volume %. The balance of the liquid phase medium may be composed of the liquid alkane(s) (with ozone and any catalyst, if present, dissolved therein). As demonstrated in the Examples, below, O₃ utilization in isobutane oxidation towards tent-butyl alcohol formation increased by 1.5-fold as the amount of water increased from 1.38 to 27.65 mmol. (See also, FIG. 2.) Other experiments showed that O₃ utilization in isobutane oxidation was in a range of from 3% to 28% in the absence of water, but increased to a range of from 40% to 150% in the presence of water (otherwise using the same conditions). This is a substantial and unexpected increase.

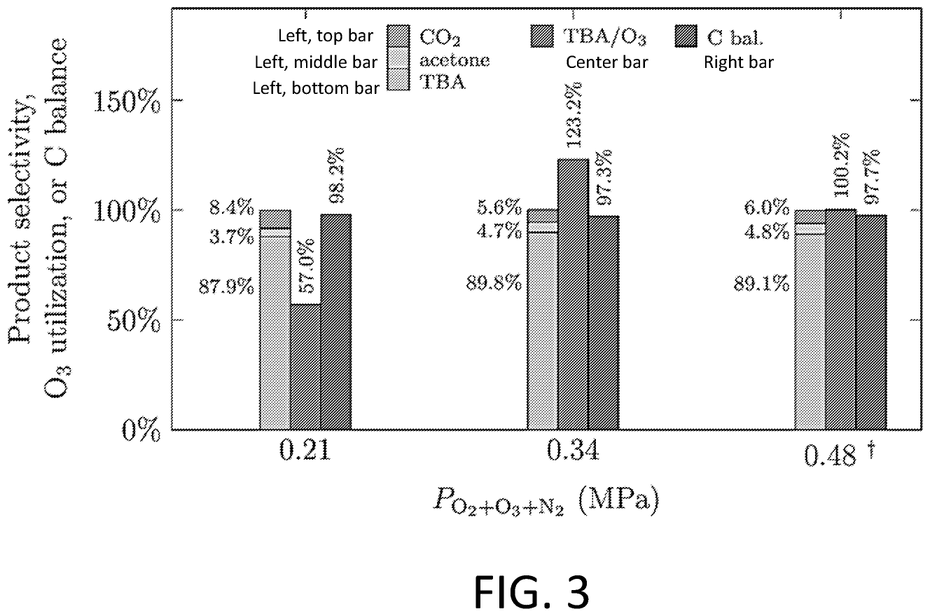

The conditions also refer to the partial pressure of non-condensable gases in the vapor phase present above, and in contact with, the liquid phase medium at the selected temperature and total pressure. For example, if the feed gas mixture comprises O₃, O₂, and N₂, the partial pressure of non-condensable gases refers to the pressure of these three gases. The partial pressure is generally selected to maximize O₃ utilization. Illustrative partial pressures include from 0.02 MPa to 0.24 MPa, from 0.1 MPa to 0.6 MPa, from 0.2 MPa to 0.5 MPa, and from 0.2 MPa to 0.4 MPa. (See also, FIG. 3.)

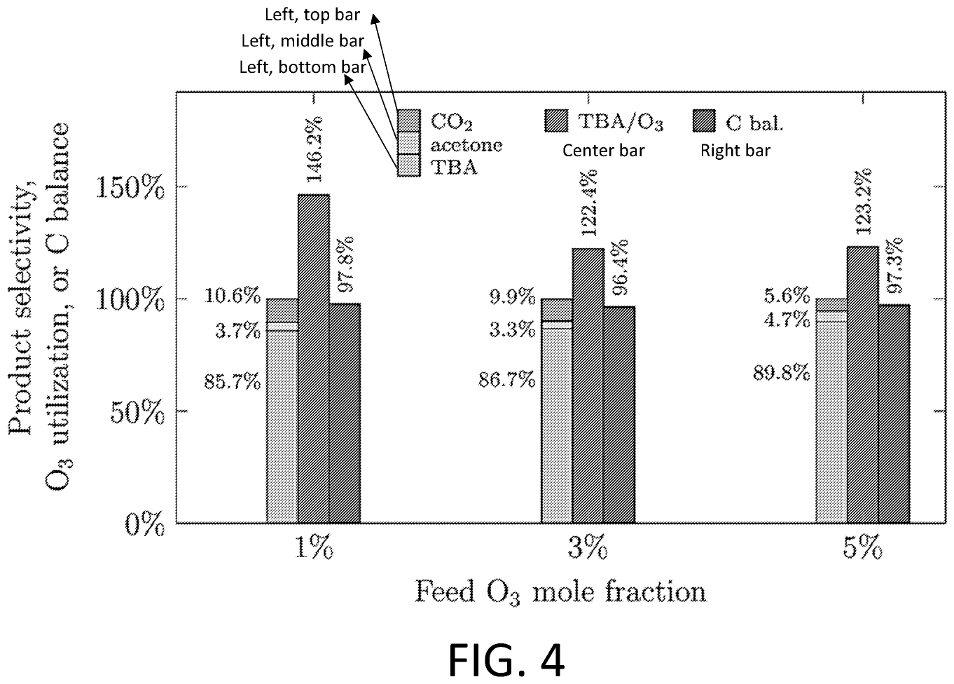

The conditions also refer to the mole fraction of ozone in the feed gas mixture. For example, if the feed gas mixture comprises O₃ in O₂ and N₂, the mole fraction refers to the O₃/(O₃+O₂+N₂) mole fraction. The mole fraction is generally selected to maximize O₃ utilization. Illustrative mole fractions of ozone include from 1% to 5%, 1.5% to 4.5%, from 2% to 4%. (See also, FIG. 4.)

The products obtained from the oxidative conversion depend upon the selected alkane as well as the conditions used. By way of example, however, isobutane may be oxidized to one or more of the following products: CO₂ (a combustion product), formic acid, acetic acid, acetone, hydroxyacetone, tent-butyl alcohol, and tent-butyl hydroperoxide. The present processes may further comprise collecting one or more of the products produced. Other steps include using the collected products, e.g., in chemical reactions to form other products. By way of illustration, tent-butyl alcohol may be dehydrated to form isobutylene.

The present processes may be characterized by a selectivity for a particular product. By selectivity, it is meant ((moles of particular product)/(total moles of products obtained))*100. In embodiments, the selectivity of the hydroxylate product is at least 80%, at least 85%, or at least 90%. For the conversion of isobutane, the hydroxylate product and these selectivities may refer to tent-butyl alcohol. Moreover, the present processes involving the conversion of isobutane may be characterized by the absence of certain products such as formic acid, acetic acid, and tent-butyl hydroperoxide (i.e., selectivity of less than 2%, less than 1%, less than 0.1%, or less than 0.05%). Finally, the present processes involving the conversion of isobutane may be characterized by a CO₂ selectivity of no more than 10%, no more than 8%, no more than 6%, or in a range of from 5% to 10%.

The present processes may be characterized by an O₃ utilization value which may be quantified as the number of moles of a desired product (e.g., hydroxylate product)/moles of ozone fed. In embodiments, the O₃ utilization value is at least 100%, at least 125%, at least 150%, at least 200%, or in a range of from 100% to 300%.

The selectivities and O₃ utilization values described above may be reported with reference to a particular set of conditions used in the process.

### EXAMPLES

### Example 1

### Introduction

In this Example, isobutane ozonation in a liquid phase was investigated using a Teflon-lined Parr reactor to avoid metal surface-induced O₃ decomposition. The Example is based, at least in part, on the insight that liquid isobutane, which possesses some compressibility at near-ambient temperatures, may be able to accommodate O₃ at higher concentrations as compared to traditional organic solvents. Another insight is the potential to leverage the pressure-tunability of O₃ concentration in liquid isobutane. Another advantage of liquid phase operation is the ability to easily maintain the isobutane-rich vapor phase above the upper flammability envelope. It is demonstrated herein that isobutane undergoes facile ozonation in its liquid phase at near ambient temperature, directly producing TBA at ˜90% selectivity. The experimental results strongly suggest that isobutane hydroxylation to TBA proceeds via the formation of the isobutane hydrotrioxide intermediate that, unlike ozonides that form during alkene ozonolysis, decomposes easily to form TBA without major release of energy.

### Experimental

### Safety

Reactions with ozone should be handled in a well-ventilated area. Condensation of ozone (at ˜112° C.) must be avoided when trapping the volatile organics using a coolant bath, to prevent potential detonation vaporization or ignition.

Isobutane most likely undergoes hydroxylation via hydrotrioxide intermediate with much lower decomposition energy compared to ozonides formed during alkene ozonolysis. However, during initial tests, the products must be handled with care because of the potential formation of explosive and shock-sensitive compounds, such as acetone peroxide from acetone and H₂O₂.

As for the O₂+O₃ gas feed in the reaction, the mixture composition should be above the upper flammable limit of 48% at low pressures. The O₂/O₃ gas feed was diluted by N₂ to shrink the flammable envelope for parametric studies involving higher pressures than a few bar. Dilution by 50% N₂ was estimated to shift the upper flammable limit from 48% to 31%.

Peng-Robinson model with Wong-Sandler mixing rules as implemented in Aspen Plus was also used to estimate the worst-case scenario with the gas phase as a closed system. While forming CO₂ and H₂O may release the most chemical energy, forming CO and H₂ will generate the largest number of molecules. The upper bound for gas phase pressure was estimated to be 7.5 MPa (forming H₂ and CO₂), which is substantially less than the Parr reactor's rating of 20 MPa.

### Reaction and Analysis Procedure

### Reactor Setup

Prior to any run, the reactor was evacuated at ˜80° C. under vacuum for ˜45min, in order to remove any residual water, acetone or tent-butyl alcohol from the reactor head. The reactor body was isolated from the liquid phase by a Teflon sleeve, to minimize ozone decomposition on the metal's (dense oxide) surface. The water used in these runs was distilled to remove compounds exhibiting acidity (pH=4-6) from the commercial supplies.

The saturation vapor pressures of liquid pure components involved in this Example at various temperatures were reviewed. Temperatures as low as −60-50° C. could condense the majority of isobutane vapor. In the −20-−10° C. range, the vapor pressure of isobutane is at least 10-times greater than those of other compounds, which allows vaporizing isobutane without losing any more than 1% of either solvents (except CH₂Cl₂) or products. In the non-ideal mixtures, the interactions between polar solvents and oxygenates further decrease the volatility of the products. These considerations determined the temperatures in the procedures for sample collection and analysis in the following sections.

### Batch Run

Batch runs were carried out at the ambient temperature in the laboratory, 17-18.5° C., (isobutane saturation pressure=0.27-0.28 MPa). The external reservoir was filled with an O₃/O₂ mixture containing 4% mol O₃/mol (O₂+O₃). The desired amount of water was optionally loaded into the sample loop and flushed into the reactor along with 20-40 mL liquid isobutane (Matheson, Ultra High Purity), which was metered by a Teledyne ISCO pump at 17° C. and 0.86 MPa (density=7.576 mol/L). The reactor content was stirred at 1000 rpm.

Seven cycles were carried out as follows: The O₃/O₂ mixture was charged to raise the reactor pressure to 0.42 MPa (introducing ˜0.08 mmol O₃), so that the gas phase contains ≥65% isobutane, above the upper flammable limit of 48%. After ˜30 min, the reactor was depressurized until the reactor pressure was close to isobutane saturation pressure. The reactor temperature was allowed to warm back to ambient temperature for ˜10 min.

After reaction, the reactor and its contents were cooled to −17° C. to reduce the vapor pressures of the products. The cold reactor was then opened to add one cold solvent among methanol (Fisher, 99.9%), dichloromethane (Acros Organics, 99.9%), and n-propyl acetate (Acros Organics, 99%). The isobutane was slowly vaporized leaving behind liquid products dissolved in the solvent.

### Batch Runs at Various Times

The reaction was carried out similarly to usual batch runs. At the end of the desired batch time, the reactor was quickly depressurized to isobutane saturation pressure at the reaction temperature, releasing the gas into a gas bag. Then the reactor was immediately moved into the freezer, using liquid nitrogen to accelerate the quench.

### Semi-Batch Run

The preparation was similar to that for the batch run. The O₃/O₂ mixture was diluted with N₂ to a molar ratio of (O₃+O₂)/N₂=1, to shrink the flammability envelope for gas-phase mixtures in the reactor.

The reaction was initiated by pressurizing the reactor with the O₃/O₂/N₂ mixture, followed by immediately opening the outlet valve and tuning a downstream rotameter to the desired set-point. A separate setup with mass flow controllers was used to calibrate the set-point for the desired flow rate with each effluent composition. The effluent passed through a condenser immersed in ethylene glycol/water mixture cooled to −60-−50° C. by liquid nitrogen, in order to trap isobutane and concentrate the non-condensables (CO₂, O₂, N₂) to be collected in a gas bag. The isobutane lost in the reactor effluent was periodically replenished.

Following the reaction, the reactor was opened to recover the liquid product. The reactor was then rinsed with a solvent (mixed with a predetermined mole fraction of internal standard, hexane). The quantities of products sticking to the Teflon sleeve were calculated from the unrecovered quantities of solvent and internal standard used for rinsing.

### Analysis

The products identified in the product mixtures from this Example include carbon dioxide, formic acid, acetic acid, acetone, hydroxyacetone, tent-butyl alcohol and tent-butyl hydroperoxide.

Gas chromatography analyses of liquid samples were done on an HP 5890 GC with its FID connected to an RXI guard column (5 m×0.53 mm) and an HP-PLOT/Q column (30 m×0.53 mm×40 μm), with injector heated to 200° C. and detector heated to 300° C. A constant ramping oven program from 30° C. to 240° C. at 10° C./min was adopted for (a) resolution between isobutane and methanol peaks, (b) resolution between hexane, propyl acetate and acetic acid, and (c) aiding product identification by retention indices. The gas and liquid samples were also injected on a Shimadzu GC equipped with a 2 m ShinCarbon packed column connected to its TCD, and a HP-PLOT/Q column (30 m×0.53 mm×40 μm) connected to its FID, mainly for analyzing CO, CO₂ (including their potential formation in the GC from any formaldehyde and formic acid in the liquid samples) and isobutane.

As a destructive analysis method, gas chromatography could decompose small quantities of formic acid, formaldehyde, and tent-butyl hydroperoxide, if any are present in the product mixtures.

HCOOH→CO₂+H₂ and HCHO→CO+H2

Tert-butyl hydroperoxide may undergo complex reactions in the GC instrument forming products (including methanol, acetic acid, and numerous unidentified products) distinguishable from chromatograms of samples containing negligible tent-butyl hydroperoxide. The peroxide/alcohol ratios of several product mixtures analyzed by GC and NMR methods were compared. GC analyses of samples with peroxide/alcohol <10⁻¹ should be avoided, as suggested by the deviation of the samples with low peroxide/alcohol ratios from those with high peroxide/alcohol ratios. On the other hand, when peroxide/alcohol is greater than 10², the alcohol formation could also interfere with the analysis.

Formic acid, acetic acid, and tent-butyl hydroperoxide were likely minor byproducts existing in all product mixtures. However, only a few runs produced quantities that were detectable with confidence (selectivities above 0.01% level). For runs that produced tent-butyl hydroperoxide or formic acid substantially above their detection limits, NMR spectra were used to determine their quantities in the product mixture.

In addition to GC results, ¹H NMR spectra of samples in deuterated dichloromethane (CD₂Cl₂, 99.8% D, Acros Organics) confirmed undetectable methanol. Thus, deuterated methanol (CD₃OD, 99.8% D, Sigma-Aldrich) was used for better resolution between signals, with dichloromethane (CH₂Cl₂) as internal standard. Neither formaldehyde nor its hemi-formal with methanol could be detected by proton NMR. Another sample using deuterated acetonitrile (CD₃CN, 99.8% D, Sigma-Aldrich) was used to confirm negligible tent-butyl hydroperoxide formation.

¹H NMR experiments showed that the tent-butyl hydroperoxide in the product mixture decomposed over extended periods of time. Therefore, all spectra for observing hydroperoxide were collected within 30 min after the end of reactions.

### Definitions

For the ozonation of isobutane only, the formation of formic acid, acetic acid, and tent-butyl hydroperoxide was negligible. Therefore, the molar selectivity for product i is defined as follows:

$${S}_{i} = \frac{{n}_{i}}{{n}_{teτr - butylalcohol} + {n}_{acetone} + {n}_{{CO}_{2}}}$$

where nᵢ [mmol] is the quantity of product i detected in the product mixture.

For the supplementary experiments with isobutane/tent-butyl alcohol mixtures, the selectivity is defined as the following.

$${S}_{i} = \frac{{n}_{i}}{{n}_{tert - butylalcohol} + {n}_{acetone} + {n}_{{CO}_{2}} + {n}_{a ceticacid} + {n}_{formicacid}}$$

The carbon balance is defined as follows.

$$C = \frac{{Σ}_{i} {N}_{C , i} {n}_{i} + {n}_{C , unreacted}}{{n}_{C , 0}}$$

where N_{C,i} is the number of carbon atoms in product i; nᵢ [mol] is the quantity of product i; n_{C,unreacted} is the total carbon quantity in unreacted isobutane (iC₄) in the cold trap condensed from reactor and in gas bags; n_{C,0} is the total quantity of carbon in isobutane fed into the system.

As an example, quantities listed in Table 1 for the base experiment with 5.53 mmol water (FIG. 2). The estimated carbon balance is

$$C = \frac{( 0.745 + 0.790 + 0.0535 + 0.00208 + 0.0008 )}{1.637} = 97.2 \%$$

TABLE 1
List of carbon quantities in feed, unreacted isobutane and products.

| Entry | Carbon, mol |
|---|---|
| Feed | 1.637 |
| iC₄ in condensate | 0.745 |
| iC₄ in gas bag | 0.790 |
| tert-Butyl alcohol | 0.0535 |
| Acetone | 0.00208 |
| CO₂ | 0.00083 |

### Results and Discussion

### Batch Runs

Preliminary experiments of isobutane ozonation at −30° C. in an open vessel suggested unexpected but beneficial effects of condensed ambient moisture on the reaction. As a result, a setup described above and shown in FIG. 7 was used to systematically evaluate the effects of controlled addition of water in a closed Parr reactor. Procedural details for the batch runs are described above. Briefly, approximately 40 mL of isobutane (iC₄) at 17° C. (saturation vapor pressure=0.86 mpa) were added to the batch reactor. A known amount of water was also added initially to study its effect. The products were analyzed by GC/FID and ¹H NMR methods (analysis protocol above). Table 2 compares the liquid phase products after 7 sequential cycles of pressurization with the O₃/O₂ mixture and depressurization to the isobutane vapor pressure at 17° C. In each cycle, the O₃ depleted in the previous cycle was replenished to generate additional tent-butyl alcohol (TBA) from the excess isobutane. When water was added, ozone utilization (defined as moles of TBA formed/moles of O₃ fed) was enhanced, and the product distribution was shifted towards predominantly TBA, with the formation of only a small amount of acetone (Entry #2, Table 2). Strikingly, in the absence of added water, nearly equimolar TBA and TBHP formation was observed at significantly lower yields of these products (Entry #1, Table 1). Therefore, the remaining experiments were carried out with added water. Designed experiments confirmed that the role of water was not to aid TBHP decomposition as initially suspected. Quite unexpectedly, water appears to favor the hydrotrioxide pathway for TBA formation. This is completely different from the free radical pathway for TBHP formation via conventional O₂ oxidation of isobutane. The reaction mechanism is discussed in more detail later.

TABLE 2
Comparison of product yields with
and without initial water addition

| | | | | TBA, | Acetone, |
|---|---|---|---|---|---|
| # | Water, mmol | O₃, mmol | TBHP, mmol | mmol | mmol |
| 1 | 0 | 0.57 | 0.084 | 0.089 | 0.008 |
| 2 | 5.53 | 0.57 | — | 0.426 | 0.034 |

Conditions: T=17° C., iC₄ 40 mL (as liquid at 17° C. and 0.86 MPa in Teledyne ISCO pump), O₃ mole fraction=4% in O₃+O₂, iC₄ saturation P˜0.25 MPa, total P˜0.42 MPa (˜0.08 mmol O₃ each cycle), gas phase iC₄ concentration ≥65% (UFL=48%¹⁴), reaction time=30 min each cycle, stirring rate 1000 rpm.

To gain insights into the kinetics of TBA formation, the effect of batch time on product yield was studied via multiple batch runs of various durations under otherwise similar operating conditions as listed in Table 2. The molar product quantities were normalized to the moles of ozone fed in each batch. The end-of-run TBA profile was regressed with a kinetic model assuming a first-order dependence on O₃ concentration with isobutane in a large excess. It is implicitly assumed that the O₃ concentration in the liquid phase is linearly proportional to the O₃ partial pressure in the gas phase. This assumption is justified based on a similar trend observed in liquid CO₂. Such an analysis yielded a pseudo-first-order rate constant of ˜0.56 min⁻¹ for O₃ consumption. The minor co-products acetone and CO2 show overlapping profiles, suggesting that they resulted from the same pathway. It is proposed that these products occurred via the β-scission of the tert-butoxy radical, which would form a methyl radical and acetone. The methyl radical was most likely converted to CO₂. Clearly, the rate of the β-scission pathway is much lower than the parallel pathway to produce TBA.

### Semi-Batch Ozonation and Parametric Studies

The remarkable TBA selectivity observed in the batch runs motivated the inventors to investigate semi-continuous ozonation in which ozone was continuously fed into the reactor containing an initial charge of isobutane (˜220 mmol). The experimental procedures are discussed above. Specifically, the effects of water, O₃ partial pressure and TBA were evaluated during semi-batch runs at 18.5° C. The total gas phase pressure was maintained at 0.75 MPa, consisting of 0.27 MPa isobutane and up to 0.48 MPa of other gases (O₃, O₂ and N₂). N₂ was added as an inert component to maintain the gas phase outside the flammability envelope. The molar (O₃+O₂):N₂ ratio was 1, and the O₃ mole fraction in the (O₃+O₂+N₂) mixture was 5%. The partial pressure of the non-condensable gases (O₃+O₂+N₂) was chosen as 0.34 MPa for the base case. The liquid products were analyzed by both GC/FID and ¹H NMR methods. The carbon balances were closed within experimental error (a sample calculation is provided in Table 1).

Effect of water: As shown in FIG. 2, varying water quantities caused only slight variations in the product distributions, with the TBA selectivity being 88%-90%. However, O₃ utilization (defined as moles of TBA formed/moles of O₃ fed) towards TBA formation increased by 1.5-fold as the initial water charge was increased from 1.38 to 27.65 mmol (˜20 fold). In other words, while an increase in the initial water concentration had only a marginal effect on TBA selectivity, it greatly increased the O₃ utilization TBA formation.

Effect of O₃ partial pressure: Due to the higher O₃ decomposition rates in the liquid phase, O₃ partitioning between the gas and liquid phases dictates ozone utilization towards TBA formation. Liquid-phase O₃ solubility depends on gas phase O₃ partial pressure as well as the properties (density, polarity, etc.) of the liquid phase. The O₃ partial pressure may be varied by changing either the total pressure of the gas phase of a fixed composition or the O₃ fraction in the gas phase at a fixed total pressure. During these investigations, the initial water added was constant at 5.53 mol.

As shown in FIG. 3, varying the partial pressure of the non-condensable gas mixture at fixed O₃ mole fraction caused only marginal changes in product distribution. The ozone utilization, however, varied quite significantly, displaying a maximum at an intermediate pressure (0.34 MPa). At lower partial pressures, the ozone partitioned more into the gas phase, oxidizing some of the gas-phase isobutane to CO₂, as evidenced by the highest CO₂ selectivity at 0.21 MPa. In contrast, at the highest partial pressure investigated (0.48 MPa), the O₃ concentration in the liquid phase increased, resulting in increased O₃ decomposition rates and over-oxidation of liquid-phase products. Hence O₃ utilization decreased significantly at higher partial pressures.

When the partial pressure of the non-condensable gases was held at 0.34 MPa and the O₃ concentration was varied, a substantial increase in O₃ utilization was observed even at 1% O₃ (FIG. 4). The CO₂ selectivity increased slightly at the lower ozone partial pressure.

While the increased gas phase O₃ partitioning increased over-oxidation of gas phase isobutane, the slower O₃ decomposition in the gas phase also enhanced overall O₃ utilization towards TBA formation. Collectively, these data demonstrate that there is a pressure-tunable optimum at which the O₃ utilization is maximized.

In summary, even though gas-phase ozonation is less selective, the gas phase may serve as a desirable reservoir at low temperatures, where O₃-based combustion and decomposition are quite slow. On the other hand, increased O₃ concentrations in the liquid may promote facile decomposition in the liquid phase, which would compete with the activation of substrate C—H bonds with high bond dissociation energies (320 -450 kJ/mol). With the appropriate choice of temperature and gas-phase partial pressure, the vapor-liquid equilibrium of O₃ may be harnessed to optimize its liquid phase concentration, and thereby to minimize both O₃ decomposition and substrate over-oxidation in the liquid phase.

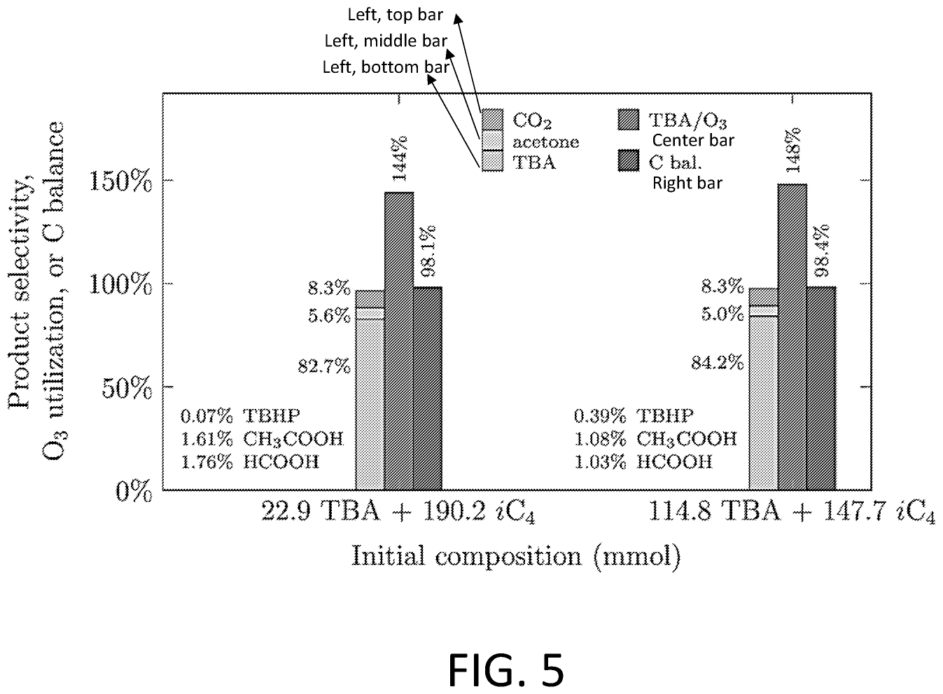

Effect of TBA in the initial mixture: The highest quantity of TBA produced in previous runs was approximately 5% of the isobutane fed to the system. It must be recognized that the isobutane is intentionally held as an excess reagent and is therefore limited by O₃ availability. In order to assess the effect of TBA accumulation on the product distribution, ozonation of mixtures of TBA+isobutane was evaluated. The net product formation (with initially added TBA subtracted) is shown in FIG. 5, with quantified minor products listed on the left side of each group. Also, traces of hydroxyacetone were detected in the GC/FID chromatogram, suggesting oxidation of the methyl group in the acetone co-product.

In general, the presence of TBA in the initial mixture promoted O₃ utilization, which is similar to the effect of added water. However, the net TBA formation in such reactions decreased. Most likely, TBA underwent oxidation to form acetic acid and formic acid with a combined selectivity of >2%. For comparison, the combined selectivity of acetic acid and formic acid was <0.02% in the isobutane ozonation runs without added TBA. Peroxyacetic acid is a possible intermediate that decomposes to acetic acid. The TBHP detected during the runs with initial TBA added was at least 10² times greater compared to previous runs with initially added water. Two factors could contribute to the observed results: (a) the solvent polarity (and hence O₃ solubility) is significantly affected as TBA is more soluble in isobutane than water; and (b) compared to water, the bulkier tent-butyl group could pose hindrance to the formation of hydrogen-bonded cyclic structures from TBA and other molecules containing hydroxyl, hydroperoxyl, or hydrotrioxyl groups.

FIG. 6, pathways a, b₁, b₂, c, d₁, d_{2,} b_{OH}, d_{OH}, summarizes plausible reaction pathways based on the observed major product spectrum. The formation of an alkyl hydrotrioxide adduct (FIG. 6, pathway a) is the first initiation step. One of the O—O bonds in the tent-butyl hydrotrioxide intermediate can cleave to give either TBHP (via pathway b₁ on FIG. 6) or TBA (via pathway b₂ on FIG. 6) via free radical formation. Based on the O₃ utilization and the observed selectivity without water addition (Table 1), pathways b₁ and b₂ on FIG. 6 likely occurred slower in the liquid phase relative to O₃ decomposition. Although Lee and Coote estimated computationally that pathway b₂ on FIG. 6 was preferred over pathway b₁ on FIG. 6 by ˜3 kcal/mol, such preference was not evident from the data in this Example (Table 2), possibly due to solvent effects not considered in the computations. (Lee, R. et al., Phys. Chem. Chem. Phys. 2016, 18 (35), 24663-24671.)

As noted above, when isobutane ozonation was performed in the presence of water or TBA, the TBA formation was substantially more selective than TBHP formation. These observations suggest a role for these protic components in aiding the decomposition of the alkyl hydrotrioxide intermediate (FIG. 6, pathway b_{OH}) to give TBA. Notably, this pathway avoids the formation of TBHP, consistent with the low formation of this product in the presence of either water or TBA. While the low TBHP yields could alternatively be explained by TBHP decomposition during the course of the reaction (FIG. 6, pathway c), the slow TBHP decomposition rates are inconsistent with the observed low peroxide selectivities. In the absence of O₃, TBHP decomposed extremely slowly following long induction periods (˜9-44 h), even in the presence of water, TBA, or acetic acid. In the presence of O₃ and water (or TBA), the TBHP decomposed more rapidly without the long induction period. However, in the presence of water, the peroxide likely also formed free radicals that promote O₃ decomposition in the liquid phase, resulting in lower ozone utilization (Table 4).

Due to the competing O₃ decomposition, the TBA formation per mole of consumed O₃ cannot surpass unity based on considering only the two pathways for unimolecular hydrotrioxide decomposition (FIG. 6, pathways b₁ and b₂). Thus, the observed TBA/O₃ ratios of up to 1.5 suggest alternative pathways including hydrotrioxide decomposition¹⁸ (via pathway d_{OH} on FIG. 6) and ROOOH formation via solvated hydrotrioxide intermediates. Also, water may promote several intermolecular reactions among ROOOH, HOOOH, HOOH, and HOH, producing caged radical pairs to aid alkane activation. While such complexes were observed at lower temperatures, the absence of similar signals in ¹H NMR spectra in this Example suggests that they are potential active species that are involved in promoting O₃ utilization. Thus, the progressive accumulation of TBA and water (as products) during the semi-continuous runs will promote alternative pathways and eventually completely inhibit TBA formation.

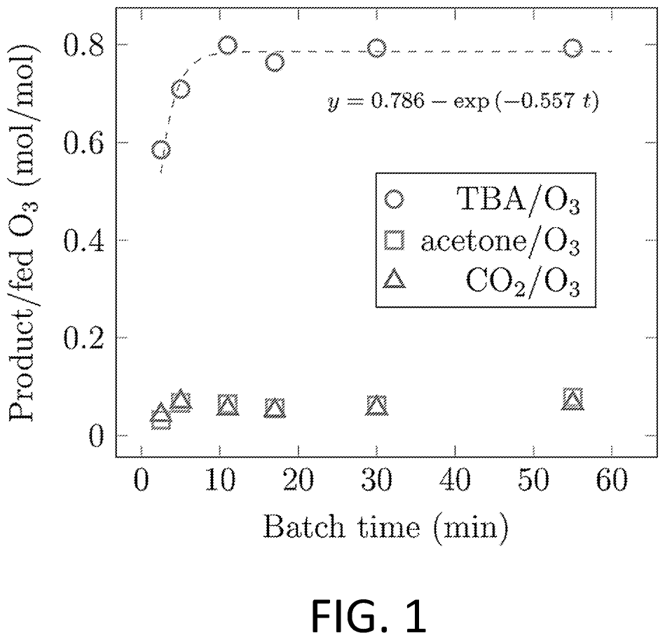

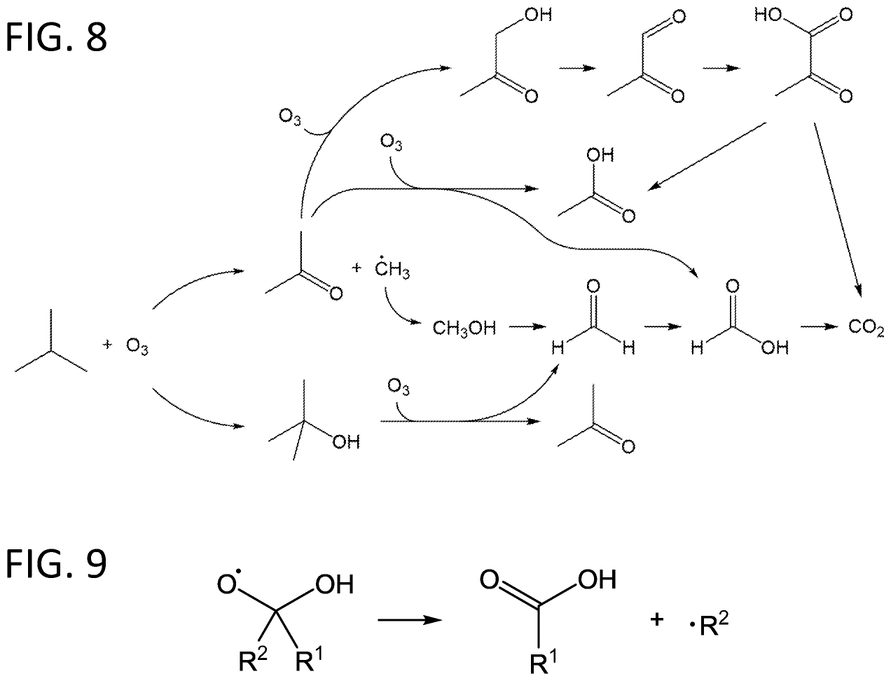

Byproducts including acetone, acetic acid, formic acid, and CO₂ formed during all runs, while hydroxyacetone and methanol were detected only under certain reaction conditions. At low ozone exposure (i.e., at short contact time and low O₃ partial pressures), nearly equimolar amounts of acetone and CO₂ (FIG. 1) are formed, suggesting that even in the presence of water, ft-scission of a tert-butoxyl radical (FIG. 6, pathway di) can still occur. However, the low yields of these products suggest that this pathway is a minor contributor. At higher ozone exposure (i.e., longer contact time and higher O₃ partial pressures), over-oxidation in the liquid-phase (suggested by FIG. 4 and Table 3) along with ozonation of gas-phase isobutane to CO₂ diminish carbon economy. A number of oxygenate oxidation pathways, shown in FIG. 8, are likely the major contributors under these conditions, forming acetic acid, formic acid, and CO₂.

The foregoing results suggest that in order to maximize TBA yield and O₃ utilization, reactor operation in which the gas and liquid phases are continuously admitted and withdrawn is needed to control the O₃ partial pressure in the gas phase as well the TBA/water accumulation in the liquid phase.

### SUMMARY

This Example demonstrates facile isobutane ozonation with 85%-90% TBA selectivity in a biphasic gas-liquid process at room temperature (16-18.5° C.) and mild pressures (˜0.6 MPa). The process could be operated safely with a non-flammable vapor phase that is well above the upper flammability limit. In contrast to oxidation with O₂ (the conventional industrial process), hydroperoxide formation was drastically reduced. It was suggested that the reduced formation of such products results from an alternate reaction mechanism when protic compounds and O₃ co-exist. With O₃, conventional free radical initiators like TBHP and DTBP are not required to initiate the reaction. In fact, such free radical initiators promote O₃ decomposition. Isobutane most likely undergoes hydroxylation via an alkyl hydrotrioxide intermediate that decomposes releasing much less energy compared to ozonides formed during alkene ozonolysis.

The O₃ utilization to form TBA is maximized by (a) an optimum concentration of protic compounds, such as water and alcohols in the liquid phase; and (b) an optimum O₃ partial pressure in the gas phase. Beyond the optimum concentration of TBA and water, undesired free-radical pathways adversely affect product selectivity. Similarly, beyond the optimum gas-phase O₃ partial pressure, the increased liquid phase O₃ concentrations promote O₃ decomposition as well as over-oxidation of TBA. Even though TBA selectivity is 85-90% during the first 2-3 h, TBA is susceptible to further oxidation upon prolonged contact with ozone. Hence, to enhance overall atom economy, continuous reactor operation is useful to operate the gas phase at the optimum O₃ partial pressure and to optimize the O₃ contact time with the liquid phase in order to simultaneously maximize O₃ utilization and TBA (desired product) yield, avoiding over-oxidation involving C—C bond cleavage. The proposed method may be extended to activating higher alkanes with tertiary carbons where the C—H bonds are more susceptible to ozonation than those on the primary carbons.

### Ozonation of Tert-Butyl Alcohol and Acetone Mixture

The commercially available tert-butyl alcohol (Sigma-Aldrich, 99.7%, b.p. 23.2° C.) is solid at a typical reaction temperature of 17° C. However, as suggested by the reaction products, a mixture of tert-butyl alcohol/acetone 17 is liquid around 17° C.

In order to evaluate the over-oxidation of tert-butyl alcohol/acetone mixture by ozone, semi-batch runs with such initial mixtures were carried out with the same average residence time of 5.2 min as the isobutane ozonation semi-batch runs. Likely due to lower ozone solubility in these liquid mixtures than in those containing isobutane, the ozone consumption was significantly less during these runs. Therefore, a catalyst bed of Pd/KIT-6 was added at the reactor outlet to decompose the unreacted ozone and to avoid its reaction with the gas bag material.

Listed in Table 3 are results from the ozonation of tert-butyl alcohol/acetone mixture analyzed by NMR experiments. In particular, ¹H NMR results confirmed no detectable oxidation of tert-butyl alcohol to tert-butyl hydroperoxide. In contrast to all other results, with less ozone exposure, the nearly zero net-formation of acetone suggests that while acetone may form from ozonation reactions, it may also be ozonized. Also, the detection of methanol suggests that it could be an intermediate that was fast oxidized to formic acid and CO₂ over longer exposure to ozone. While tert-butyl alcohol and acetone should be more prone to oxidation than isobutane, the carbon increments in the last column (estimated to be the carbons from tent-butyl alcohol) suggest ozone utilization is less than that during isobutane ozonation. It could be inferred that the reaction rate in such isobutane-absent mixtures was likely around 10⁻¹ of that in isobutane-rich mixtures. Due to the low vapor pressure of the mixture (˜3.4 kPa), over-oxidation in gas-phase contributed dramatically less than isobutane (P=0.25-0.27 MPa) ozonation. Therefore, the results should represent the oxygenate over-oxidation in the liquid phase.

TABLE 3
Products from the ozonation of tert-butyl alcohol/acetone mixture

| O₃, | Reaction | Acetone, | Acetic | CH₃OH, | HCOOH, | CO₂, | ΔC/4, |
|---|---|---|---|---|---|---|---|
| mmol | time, min | mmol | acid, mmol | mmol | mmol | mmol | mmol |
| 5.5 | 61 | −0.06 | 0.23 | 0.53 | 0.072 | 0.12 | 0.25 |
| 11.2 | 124 | +0.97 | 0.28 | — | 0.33 | 1.04 | 0.64 |
| 12.7 | 141 | +0.99 | 0.38 | — | 0.32 | 1.06 | 0.71 |

Conditions: Initial mixture of 148.4 mmol TBA + 10.49 mmol acetone, T = 18.0° C., 5% O₃/(O₃ + O₂ + N₂), P(O₃ + O₂ + N₂) = 0.34 MPa.
Note:
The acetone column represents the net formation, i.e., with initial quantities subtracted from the final measurements. ΔC represents the sum of carbons of all the net product formation. Assuming closed carbon balance, ΔC/4 estimates the quantities of oxidized tert-butyl alcohol.

Investigation into Tert-Butyl Hydroperoxide Decomposition

For the majority of the semi-batch runs, the final selectivities towards tert-butyl hydroperoxide were less than 0.02%. However, those runs could not provide evidence whether (a) the OH-containing compounds (water and tert-butyl alcohol) promoted the peroxide decomposition, or (b) the formation of peroxide was inhibited. Therefore, tert-butyl hydroperoxide decomposition was evaluated in various environments to elucidate the mechanisms for the observed low peroxide production.

### Effect of Acetic Acid

While tert-butyl hydroperoxide decomposition occurs in product mixtures, exactly overlapping spectra were collected from a sample of tert-butyl hydroperoxide/nonane within 12 h after adding a drop of acetic acid (glacial, Fisher). By 44 h after adding acetic acid of 1.5 orders of magnitude higher concentration than that detected in the product mixture, only 4.4% tent-butyl hydroperoxide was converted. Such slow process indicates that acetic acid cannot contribute to either the low peroxide production or the post-reaction hydroperoxide decomposition during the 2-3 h runs. Also, as confirmed experimentally, acetic acid alone does not significantly affect tent-butyl hydroperoxide reactions in the GC instrument.

The co-existence of water and acetic acid may potentially promote tent-butyl hydroperoxide conversion with water, aiding the dissociation of acetic acid. However, mixtures of tent-butyl hydroperoxide/nonane+acetic acid+water in CD₃CN likely contained protons that were excessively active for observation with proton NMR.

### Effect of Ozonation of Typical Reactant/Product Mixtures

The proton NMR spectra were compared from samples before and after treating a mixture of tent-butyl alcohol+acetone+tert-butyl hydroperoxide+water with ozone for 66 min. While the —OOH signal decreased by 13.7% and broadened after ozonation, no tent-butyl hydroperoxide decomposition in the no ozonation sample could be detected 24 h after spectrum collection. The contrast confirmed that tent-butyl hydroperoxide was decomposed by ozonation, but the process was rather slow in the polar solution, possibly due to the lower ozone solubility in absence of isobutane.

The ozonation of tent-butyl hydroperoxide/water/isobutane mixtures with various initial peroxide content (TBHPᵢₙ column) is shown in Table 4. The last column (TBA_{oz}) estimates the tent-butyl alcohol formed from isobutane ozonation, by subtracting the quantity of converted hydroperoxide from the total quantity of alcohol. Even though the product selectivity from hydroperoxide decomposition is unknown, the low acetone formation also confirms the detrimental effect of hydroperoxide addition to ozone utilization, even at a hydroperoxide/isobutane ratio as low as 0.04%. The results suggest that the hydroperoxide and ozone promote the decomposition of each other. Therefore, the hydroperoxide formation at the initial stages of isobutane ozonation should be low to achieve the high ozone utilization observed in FIGS. 2-4.

In summary, two mechanisms together appear to contribute to the observed low tent-butyl hydroperoxide during isobutane ozonation: (a) in the presence of OH-containing compounds (such as water, acetic acid, and tent-butyl alcohol), tent-butyl hydroperoxide formation is less competitive than alternative pathways, which consumed the hydrotrioxide intermediate faster, and (b) the decomposition of the drastically less peroxide is also promoted by ozonation. As the product accumulates, the solvent properties (including polarity, coordination, and ozone solubility) change with the solution composition, and alter the selectivities among the decomposition pathways of the hydrotrioxide intermediates.

TABLE 4
Ozonation products from tert-butyl hydroperoxide/water/isobutane
mixture

| TBHPᵢₙ, | TBHPₒᵤₜ, | TBA, | Acetone, | Acetic | CO₂, | TBA_{oz}, |
|---|---|---|---|---|---|---|
| mmol | mmol | mmol | mmol | acid, mmol | mmol | mmol |
| 0 | <0.01 | 6.40 | 0.30 | <0.02 | 0.48 | 6.40 |
| 0.108 | <0.01 | 3.73 | 0.17 | 0.09 | 0.28 | 3.62 |
| 0.540 | 0.042 | 3.68 | 0.12 | 0.20 | 0.35 | 3.18 |
| 2.720 | 0.124 | 6.57 | 0.13 | 0.17 | 0.40 | 3.97 |

Common conditions: T = 16-17° C., initial water (including water from aqueous tert-butyl hydroperoxide solution) 27.8 mmol, isobutane 241 mmol, consumed ozone 4.32 mmol, N₂ + O₂ + O₃ gas mixture containing 5% ozone, N₂ + O₂ + O₃ partial pressure 0.34 MPa.
Note:
TBA_{oz} = TBA − TBHPᵢₙ + TBHPₒᵤₜ.

### Possible Over-Oxidation Pathways

At low ozone exposure (short contact time and low O₃ partial pressures), β-scission (FIG. 6, pathway d₁) is likely the dominant pathway for CO₂ formation. The observation of formic acid and CO₂ in most isobutane ozonation reactions suggests that the oxidation of the methyl radical is one of the plausible routes. Alternatively, in the case of H-abstraction to form methane, methane is likely susceptible to ozonation, as suggested by its ozonation in gas phase. Formic acid formation was observed by proton NMR in a sample of methanol with ozone continuously bubbled for 30 min.

Prolonged contact of oxygenates and O₃, as well as higher O₃ partial pressures, leads to the over-oxidation in the liquid-phase (suggested by FIG. 4 and Table 3). A number of oxygenate oxidation pathways, shown in FIG. 8, are likely the major contributors forming acetic acid, formic acid, and CO_{2.} Over-oxidation along with the ozonation of gas-phase isobutane to CO₂ diminish carbon economy.

The detection of hydroxyacetone indicates oxidation of terminal methyl groups, which was likely responsible for the over-oxidation forming C₁-C₃ products (for example, via the further hydroxyacetone oxidation in FIG. 8 or FIG. 9). The ozonation of TBA/acetone mixture in Table 3 suggests that while acetone could form from TBA ozonation, it may also be oxidized via C—C bond cleavage.

Methanol could possibly form (see FIG. 9), for example, from ozone-attacked acetone. Indeed, Table 3 also showed methanol detectable only at the lowest ozone exposure. It could be a short-lived intermediate that is further oxidized to formic acid and CO2 over longer exposure to ozone.

### Example 2

In this Example, experiments similar to those in Example 1 were carried out but using a higher temperature (50° C.).

### Experimental

### Chemicals

The dioxygen (UHP Plus), argon (HP), and isobutane (UHP) were all purchased from Matheson.

### Multi-Batch Runs

The procedure and experimental apparatus of these experiments have been described in detail in Example 1. The dioxygen stream was used to generate ozone by an Atlas Ozone Generator, and then mixed with argon in a reservoir. Briefly, a Teflon-lined Parr vessel was vacuumed prior to charging the desired amounts of the liquid alkane from an ISCO syringe pump cooled to 10° C. An option to direct the liquid alkane stream through a sample loop containing distilled water was provided to meter in controlled amounts of water. The reactor stirrer was set at 1000 rpm. For each of the sequential batch runs, the reactor was pressurized with an O₂/O₃/Ar gas mixture of known composition. After reaching a predetermined pressure, the gas inlet valve was shut off, and the reaction was allowed to proceed for 15 min in the closed batch reactor. Then the gas outlet was opened. The alkane that escapes with the gas phase was partially condensed in a cold trap held around −60 to −50° C. and ambient pressure to concentrate the CO₂. The gas from the condenser was collected in Tedlar sample bags. After the reactor pressure reaches the vapor pressure of the alkane mixture, and equilibrated to the room temperature, the procedure was repeated. At the end of all the sequential batch runs, the reactor was placed in an ice bath kept in a walk-in freezer at −18° C. At this temperature, the vapor pressure of all compounds remaining in the reactor is much reduced. Then a weighed amount of cold methanol was added into the reactor, and the volatile alkane was allowed to vaporize at 0-4° C. and condensed in the cold trap around −60 to −50° C. After adding 2-pentanone as an internal standard, the methanolic liquid sample was injected into a GC equipped with a HP-PLOT/Q column to resolve ≥C₂ products and a flame ionization detector (FID). The methanolic liquid sample was also added to D₂O with maleic acid as internal standard to quantify formic acid by ¹H NMR. The gas samples collected in tedlar bags were injected into another GC equipped with a thermal conductivity detector to analyze the CO₂, and a FID to analyze the hydrocarbons.

### Definitions

As isobutane oxidation is limited by the quantity of ozone, conversions are estimated with detected products from isobutane as follows, treating the CO₂ as a byproduct of acetone formation via beta-scission.

$${X}_{i {C}_{4}} = \frac{{n}_{TBA} + {n}_{acetone}}{{n}_{i {C}_{4} , 0}}$$

wherein n_{iC}_{_{4,}}₀ is the initial moles of isobutane loaded in each batch.

The selectivity for product i is defined as follows:

$${S}_{i} = \frac{{n}_{i}}{{Σ}_{j} {n}_{j}}$$

wherein the denominator represents the total moles of all products. Two measures were adopted for ozone utilization. One is ratio of (moles of TBA formed)/(initial moles of O₃). Since dioxygen alone cannot oxidize isobutane to detected products at 50° C., the following definition was used to characterize the average quantity of electron transfer induced by each mole of ozone.

$$U = \frac{( quantity of electron transferred to oxidants )}{4 \times ( quantity of {O}_{3} introduced )} = \frac{{n}_{e}}{4 {n}_{{O}_{3}}} \qquad Eq . ( 1 )$$

$${n}_{e} = 2 {n}_{tert - butanol} + 3 {n}_{acetone} + 5 {n}_{formicacid} + 7 {n}_{{CO}_{2}}$$

During the experiment, the cold trap and its contents were weighed to estimate the amount of condensed isobutane. The carbon balance is defined as follows.

$$C = \frac{\begin{matrix}amount of carbons in \\ ( condensed isobutane + products + gas samples )\end{matrix}}{amount of carbons charged into reactor}$$

$$amount of carbons in product = 4 {n}_{tert - b utanol} + 3 {n}_{acetone} + {n}_{f ormicacid} + {n}_{{CO}_{2}}$$

### Effects of Elevated Temperature on Isobutane Hydroxylation

The formation of C—C cleavage products suggests that alkoxyl free radicals may be involved in the reactions. Some secondary radicals may react with dioxygen to yield peroxyl radicals. This process could potentially be promoted at elevated temperature. However, the ozone decomposition rate will also be enhanced at higher temperatures and may pose a limitation. Interestingly, as shown in 5 (Row #1), significant increases in ozone utilization are observed for isobutane hydroxylation at 50° C. compared to the highest value of 80% (TBA/O₃=151%) around 17° C. (see Example 1) while maintaining a high TBA selectivity >87%. On average, the 5.9-11 mol of oxidizing equivalents induced by each mol of O₃ indicates that O₂ is involved in the reaction. On Row #2, the cumulative product formation of 7 sequential batch runs without removing the products from the reactor does not scale compared to the result of a single batch run on Row #1. The result suggests the ozone utilization may be inhibited by progressive alcohol formation, which is consistent with the conclusion from the empirical model.

TABLE 5
Product formation from isobutane ozonation at 50° C.

| O₃, μmol × # | | TBA, | Acetone, | CO₂, | O₃ | |
|---|---|---|---|---|---|---|
| of batches | X_{iC4} | μmol | μmol | μmol | utilization ^{a} | TBA/O₃ ^{b} |
| 74 × 1 | 0.12% | 350 ± 5 | 18 ± 2 | ~10¹ | 261% | 475% |
| 57 × 7 | 0.37% | 1050 ± 35 | 58 ± 4 | 43 ± 8 | 147% | 263% |

Conditions: 0.3 mol isobutane with 100 μL water initially, 50° C., P_{isobutane} = 0.67 MPa, O₃/(O₃ + O₂) = 5% mol/mol, P(O₃ + O₂) = 0.053 − 0.066 MPa.
^{a} Definition as in Eq. (1);
^{b} TBA/O₃ molar ratio as used in Example 1.

Other information and data may be found in United States Patent Application 63/158,522, which is hereby incorporated by reference in its entirety.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more”.

If not already included, all numeric values of parameters in the present disclosure are proceeded by the term “about” which means approximately. This encompasses those variations inherent to the measurement of the relevant parameter as understood by those of ordinary skill in the art. This also encompasses the exact value of the disclosed numeric value and values that round to the disclosed numeric value.

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

1. A process for oxidizing an alkane, the process comprising combining an alkane and ozone in a liquid phase medium comprising a protic additive under conditions sufficient to oxidize the alkane to products comprising a hydroxylate.

2. The process of claim 1, further comprising adding the protic additive to the liquid phase medium prior to oxidizing the alkane.

3. The process of claim 1, wherein the protic additive is water, an alcohol, or a combination thereof.

4. The process of claim 1, wherein the protic additive is water.

5. The process of claim 1, wherein the protic additive is present in the liquid phase medium at an amount of at least 1 mmol.

6. The process of claim 1, wherein the liquid phase medium comprises no more than 1 mmol CO₂.

7. The process of claim 1, wherein the liquid phase medium is free of added CO₂.

8. The process of claim 1, wherein the liquid phase medium comprises from 0.2 volume % to 5 volume % of the protic additive and a balance of the alkane.

9. The process of claim 1, wherein the alkane is a C2-C20 alkane.

10. The process of claim 1, wherein the alkane is a branched alkane.

11. The process of claim 1, wherein the alkane is isobutane.

12. The process of claim 1, wherein the combining is carried out at a temperature of at least 15° C. and a total pressure of a vapor phase present above the liquid phase medium of no more than 5 MPa.

13. The process of claim 1, wherein a partial pressure of non-condensable gases in a vapor phase present above the liquid phase medium is in a range of from 0.1 MPa to 0.6 MPa.

14. The process of claim 1, wherein a mole fraction of ozone in a feed gas mixture providing the ozone is in a range of from 1% to 5%.

15. The process of claim 1, wherein the process is characterized by a selectivity for a hydroxylate of at least 80%, a O₃ utilization value defined as moles of the hydroxylate produced per moles of ozone consumed of at least 100%, or both.

16. The process of claim 1, wherein the process produces CO₂ at a selectivity of no more than 10%.

17. A process for oxidizing an alkane, the process comprising combining an alkane and ozone in a liquid phase medium comprising a protic additive under conditions sufficient to oxidize the alkane to products comprising a hydroxylate, wherein the liquid phase medium is free of added CO₂, and
adding the protic additive to the liquid phase medium prior to oxidizing the alkane.

18. The process of claim 17, wherein the protic additive is water and the alkane is isobutane.

19. The process of claim 18, wherein the water is present in the liquid phase medium at an amount of at least 1 mmol.

20. The process of claim 18, wherein the liquid phase medium comprises from 0.2 volume % to 5 volume % of the water and a balance of the alkane.

21. (canceled)
